(19) United States
(12) Patent Application Publication
(10) Pub. No.: US 2006/0047226 A1
(43) Pub. Date: Mar. 2, 2006

(54) Nasal sizing device

(76) Inventor: Thomas J. Wood, Blackshear, GA (US)

Correspondence Address: OLDS, MAIER & RICHARDSON, PLLC, PO BOX 20245, ALEXANDRIA, VA, 22320-1245, US

(21) Appl. No.: 10/926,353

(22) Filed: Aug. 26, 2004

(51) Int. Cl. A61B 5/103

(52) U.S. Cl. 600/587

(57) ABSTRACT

A device for measuring a nasal cavity. The device may include a support body having a substantially flat top and a substantially flat bottom, a plurality of support arms radially disposed from said central support body and a plurality of protrusions having a substantially oval contact surface.

### BACKGROUND OF THE INVENTION

1. Field of the Invention

Exemplary embodiments of the present invention generally relate to nasal sizing devices.

2. Discussion of the Background

Obstructive sleep apnea syndrome (commonly referred to as obstructive sleep apnea, sleep apnea syndrome, and/or sleep apnea) is a medical condition which includes repeated, prolonged episodes of cessation of breathing during sleep. During a period of wakefulness, the muscles of the upper part of the throat passage of an individual keep the passage open, thereby permitting an adequate amount of oxygen to flow into the lungs. During sleep, the throat passage tends to narrow due to the relaxation of the muscles. In those individuals having a relatively normally sized throat passage, the narrowed throat passage remains open enough to continue to permit the adequate amount of oxygen to flow into the lungs. However, in those individuals having a relatively smaller sized throat passage, the narrowed throat passage prohibits the adequate amount of oxygen from flowing into the lungs. Additionally, a nasal obstruction, such as a relatively large tongue, and/or certain shapes of the palate and/or the jaw of the individual further prohibit the adequate amount of oxygen from flowing into the lungs.

The individual having the above-discussed conditions can stop breathing for one or more prolonged periods of time (e.g., 10 seconds or more). The prolonged periods of time during which breathing is stopped, or apneas, are generally followed by sudden reflexive attempts to breathe. The reflexive attempts to breathe are generally accompanied by a change from a relatively deeper stage of sleep to a relatively lighter stage of sleep. As a result, the individual suffering from obstructive sleep apnea syndrome generally experiences fragmented sleep that is not restful. The fragmented sleep results in one or more of excessive and/or inappropriate daytime drowsiness, headache, weight gain or loss, limited attention span, memory loss, poor judgment, personality changes, lethargy, inability to maintain concentration, and/or depression.

Other medical conditions can also prevent individuals, including adults and infants, from receiving the adequate amount of oxygen into the lungs. For example, an infant who is born prematurely can have lungs that are not developed to an extent necessary to receive the adequate amount of oxygen. Further, prior to, during, and/or subsequent to certain medical procedures and/or medical treatments, an individual can be unable to receive the adequate amount of oxygen.

Under these circumstances, it is known to use a ventilation interface to apply a positive pressure to the throat of the individual, thereby permitting the adequate amount of oxygen to flow into the lungs. In a known ventilation interface, oxygen and/or room air containing oxygen can be delivered through the nose of the individual via nasal inserts.

One conventional ventilation interface for the application of such positive pressures includes a cannula with nasal inserts configured to be inserted into corresponding nares of the nose.

In the conventional art, in order for a cannula with nasal inserts to work properly the nasal inserts must fit snuggly within the nostril to maintain the required seal. The seal can created be some combination deformation of the nasal insert and the individual's nostril. As a result, it is common for individuals to use an improperly fitted cannula with nasal insert resulting in suboptimal effect sealing effect due to a broken or partially broken seal when wearing the cannula while sleeping. Further, the required seal is generally difficult to maintain when the improperly sized nasal inserts are inserted into the nostrils.

The foregoing description of the related art is merely intended to illustrate one aspect of use of the invention. One skilled in the art will appreciate the multiplicity uses of the invention. For brevity sake only one example of possible usage has been recited in this background portion and therefore the foregoing description is not intended to limit the scope of the invention is provided but, is provided merely as one illustrative example for its use.

Consequently, a need still exists for a device which provides a solution to the aforementioned problem in the background art.

### SUMMARY OF THE EXEMPLARY EMBODIMENTS

Exemplary embodiments of the present invention are directed to a device for measuring a nasal cavity. The device may include a support body having a flat top and a flat bottom, a plurality of support arms radially disposed from said central support body and a plurality of protrusions having a substantially oval contact surface.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 is a top perspective view of an exemplary embodiment of a nasal measuring device according to the present invention.

FIG. 2 is a top plan view of an exemplary embodiment of a nasal measuring device according to the present invention.

FIG. 3 is bottom perspective view of an exemplary embodiment of a nasal measuring device according to the present invention.

FIG. 4 is a side elevational view of an exemplary embodiment of a nasal measuring device according to the present invention.

### DETAILED DESCRIPTION OF THE PREFERRED EMBODIMENTS

Referring now to the drawings, wherein like reference numerals designate identical or corresponding parts throughout the several views.

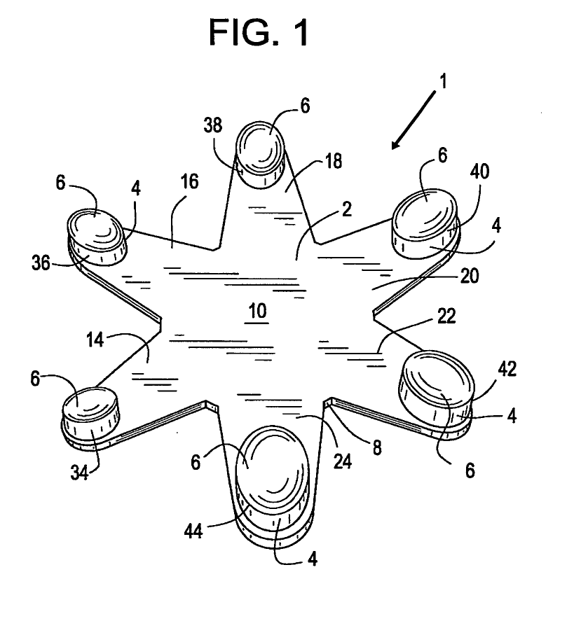

Referring now particularly to FIGS. 1-3, there is illustrated one embodiment of a nasal measuring device 1 with a flat top side 10. One embodiment is directed to a device for measuring a nasal cavity. The device 1 may include a support body 2 having a flat top 10 and a flat bottom 12, a plurality of support arms radially disposed from said central support body 2 and a plurality of protrusions 34, 36, 38, 40, 42 and 44 each having a oval top contact surface 6 and a side contact surface 4. The side wall 8 of the support body 2 interposed between the plurality of support arms has a side wall 8 having a flat surface and a substantially constant thickness. In one embodiment, the side wall 8 may be about 1-5 mm thick.

FIG. 1 of the nasal measuring device 1 on the flat top side 10 includes a support body 2 with a plurality of support arms 14, 16, 18, 20, 22 and 24 disposed radially from the support body 2. Each of the plurality of support arms 14, 16, 18, 20, 22 and 24 has protrusions 34, 36, 38, 40, 42, and 44, respectively positioned on the distal end of the support arm.

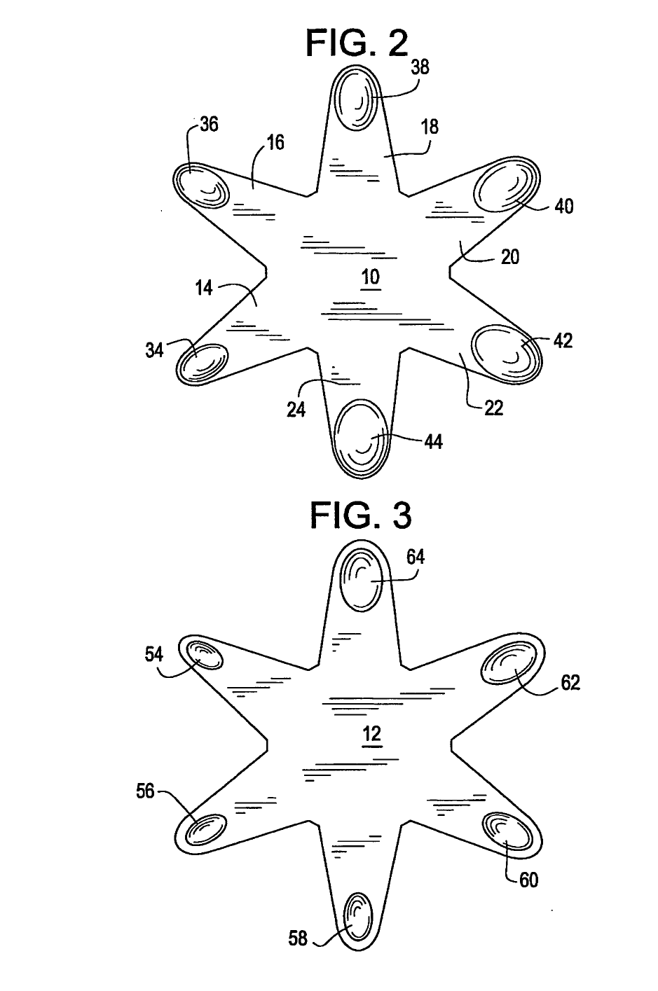

FIG. 2 shows a top down view of the device 1 of a top surface 10. The extra small support arm 14 has an extra small protrusion 34 on the distal end of the extra small support arm 14. Likewise, the small support arm 16 has a small protrusion 34 on the distal end of the small support arm 16 and the medium support arm 18 has a medium protrusion 38 on the distal end. Moving in a clockwise direction around the embodiment shown in FIG. 2, the medium plus support arm 20 has a medium plus protrusion 40 on the distal end. Similarly, the large support arm 22 has a large protrusion 42 positioned on the distal end. Lastly, the extra large support arm 24 has an extra large protrusion 44 positioned on the distal end.

In an exemplary embodiment the extra small protrusion 34 may be, for example, about 9-14 mm in length or shorter and have a width of about 8-12 mm or shorter. Likewise, the small protrusion 34 may be about 12-16 mm in length and about 10-14 mm in width, as a non-limiting example. In an exemplary embodiment, the medium protrusion 38 may be about 12-17 mm in length and have a width of about 11-14 mm. Similarly, the medium plus protrusion 40 may be about 16-20 mm in length and about 13-17 mm in width, as a non-limiting example. Lastly, in an exemplary embodiment the large protrusion 42 may be about 19-23 mm in length and about 14-18 mm in width and the extra large protrusion 44 about 21-25 mm in length or longer and about 16-20 mm in width or wider. These ranges are considered exemplary and one of ordinary skill in the art will appreciate that upper and lower ranges of the protrusions may exceed or be smaller than the ranges list above and, in particular, may be adapted to the use of the device (e.g. the size of the individual/mammal being measured)

FIG. 3 shows a bottom view of the nasal measuring device having a bottom surface 12. The extra small support arm 14 has an extra small hollow cavity 54 on the distal end of the extra small support arm 14. Likewise, the small support arm 16 has a small hollow cavity 56 on the distal end of the small support arm 16 and the medium support arm 18 has a medium hollow cavity 58 on the distal end. Moving in a counter-clockwise direction around the exemplary embodiment shown in FIG. 3, the medium plus support arm 20 has a medium plus hollow cavity 60 on the distal end. Similarly, the large support arm 22 has a large hollow cavity 62 positioned on the distal end. Lastly, the extra large support arm 24 has an extra large hollow cavity 64 positioned on the distal end.

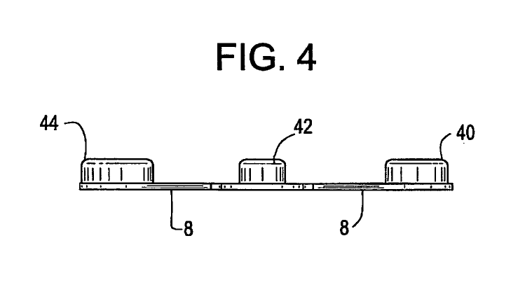

FIG. 4 shows a side view of the nasal measuring device. As illustrated, the side wall 8 of the nasal measuring device maintains substantially consistent thickness throughout the several sides. As discussed above, the side wall 8 may be about 1-5 mm, as one non-limiting example. FIG. 4 shows an extra large protrusion 44 on the left side with a large protrusion 42 in the middle and the medium plus protrusion 40 on the right side of the device.

FIGS. 1-4 generally illustrate an exemplary embodiment of a nasal measuring device. One skilled in the art would appreciate that the device may be reconfigured to of various sizes to accommodate, for example, infants or adults with different sized nasal cavities. Likewise, one skilled in the art would appreciate that device may be tailored and resized to accommodate different users with different physical requirements which may require smaller or larger protrusions to accurately size one for a nasal insert type CPAP device or another other clinical device that may require nasal cavity measurements. An exemplary embodiment of the present invention provides that a user or physician may hold the nasal measuring device by the support arm and gently insert or attempt to insert each protrusion into a patient's nose until determining which protrusion 34, 36, 38, 40, 42 or 44 that creates the best fit and consults the corresponding chart to determine, for example, which size nasal inserts would be best suited for the user.

Further, the specific dimensions of each protrusion are configured to correspondingly relate in size to a nasal insert device (e.g. cannula with nasal inserts connected to a CPAP ventilation machine). The nasal measuring device in an exemplary embodiment of the present invention has substantially oval shaped cavities 54, 56, 58, 60, 62, 64 with contact portions that may be configured to be engageable with a user's nostril. Further, the contact elements are disposed on a support body 2 in a way that provides substantial symmetry and consistency in nostril contact.

Numerous modifications and variations of the present invention are possible in light of the above teachings. It is therefore to be understood that within the scope of the appended claims, the invention may be practiced otherwise than as specifically described herein.

1. A device for measuring a nasal cavity, comprising:
a central support body having a substantially flat top and a substantially flat bottom, a plurality of support arms radially disposed from said central support body; and
a plurality of protrusions each having a substantially oval contact surface disposed at outer ends of said support arms.

2. The device of claim 1 wherein said central support body has a substantially flat top and a substantially flat bottom,

3. The device of claim 1 wherein said plurality of protrusions are at least two different sizes.

4. The device of claim 1 wherein said plurality of protrusions are at least three different sizes.

5. The device of claim 1 wherein said support arms are substantially the same thickness.

6. The device of claim 1 wherein said plurality of protrusions are substantially symmetrical in relation to the center of said support body.

7. The device of claim 1 wherein said plurality of protrusions define a plurality of hollow cavities formed on substantially flat bottoms of said plurality of support arms.

8. The device of claim 7 wherein said plurality of hollow cavities are at least two different sizes.

9. The device of claim 7 wherein said plurality of hollow cavities are at least three different sizes.

10. The device of claim 1 wherein said support body, said plurality of support arms and said plurality of protrusions are substantially opaque.

11. The device of claim 1 wherein said plurality support arms have substantially the same width.

12. The device of claim 1 wherein at least one of said central support body, said plurality of support arms and said plurality of protrusions are slightly pliable.

13. A sizing device for a nasal inserts comprising:
a central support body having a substantially flat top and a substantially flat bottom, a plurality of support arms radially disposed from said central support body; and
a plurality of protrusions each having a substantially oval contact surface;
wherein said plurality of protrusions are configured to accommodate a range of physical characteristics of a nasal cavities.

14. The sizing device for a nasal inserts according to claim 13 wherein the range of physical characteristics of a nasal cavity are determined by age of an individual.

15. The sizing device for a nasal inserts according to claim 13 wherein the range of physical characteristics is determined by at least one of weight and height of an individual.

16. The device of claim 13, with the means for measuring the size of a nasal cavity that is configured to correspond to a range of nasal insert devices.

17. The device of claim 16, wherein the nasal insert devices are CPAP devices.

18. A device for measuring a nasal cavity, comprising:
a central support body having a substantially flat top and a substantially flat bottom, a plurality of support arms radially disposed from said central support body; and
means for measuring the size a nasal cavity.

19. The device of claim 18, wherein the means for measuring the size of a nasal cavity is configured to correspond to a range of nasal insert devices.

20. The device of claim 19, wherein the nasal insert devices are CPAP devices.
